(19) United States
(12) Patent Application Publication
(10) Pub. No.: US 2023/0234901 A1
(43) Pub. Date: Jul. 27, 2023

(54) PREPARATION OF AN IMPROVED COMPOSITION FROM 1-CHLORO-3,3,3-TRIFLUOROPROPENE (HFO-1233ZD) HIGH BOILING RESIDUE BY-PRODUCT

(71) Applicant: Honeywell International Inc., Charlotte, NC (US)

(72) Inventors: Gustavo Cerri, Parsippany, NJ (US); Jinhua Yao, Shanghai (CN)

(21) Appl. No.: 18/083,237

(22) Filed: Dec. 16, 2022

Related U.S. Application Data
(60) Provisional application No. 63/331,970, filed on Apr. 18, 2022.

(30) Foreign Application Priority Data
Jan. 26, 2022 (WO) PCT/CN2022/073913

(51) Int. Cl. C07C 17/20; C09K 5/04

(52) U.S. Cl. CPC C07C 17/204; C09K 5/045

(57) ABSTRACT

High boiling fluorinated by-products obtained from a manufacturing process of 1-chloro-3,3,3-trifluoropropene (HFO-1233zd) not only include components that can be used as a starting material or feedstock in the production of 1,1,1,3,3-pentafluoropropane (HFC-245fa), but also contain impurities that can be detrimental in the HFC-245fa process. A method of providing an improved composition from the high boiling by-products obtained from an HFO-1233zd manufacturing process reduces these impurities. The improved composition can be used as a starting material or feedstock for the production of HFC-245fa.

### CROSS-REFERENCE TO RELATED APPLICATION

This application claims priority to International Patent Application No. PCT/CN/2022/073913, filed Jan. 26, 2022, and U.S. Provisional Patent Application No. 63/331,970, filed Apr. 18, 2022, both of which are herein incorporated by reference in their entireties.

### FIELD

The present disclosure is related to a process for preparing an improved composition comprising fluorinated by-products from a process for manufacturing 1-chloro-3,3,3-trifluoropropene (HFO-1233zd). This improved composition may be used in the production of 1,1,1,3,3-pentafluoropropane (HFC-245fa).

### BACKGROUND

Chlorofluorocarbons (CFCs) such as trichlorofluoromethane and dichlorodifluoromethane have been used as refrigerants, blowing agents and diluents for gaseous sterilization. In recent years, there has been widespread concern that certain chlorofluorocarbons might be detrimental to the Earth's ozone layer. As a result, there is a worldwide effort to use halocarbons which contain fewer or no chlorine substituents. Accordingly, the production of hydrofluorocarbons, or compounds containing only carbon, hydrogen and fluorine, has been the subject of increasing interest to provide environmentally desirable products for use as solvents, blowing agents, refrigerants, cleaning agents, aerosol propellants, heat transfer media, dielectrics, fire extinguishing compositions, power cycle working fluids, and starting material for producing hydrofluoroolefins that do not harm the ozone layer and also have low global warming potential.

In this regard, 1,1,1,3,3-pentafluoropropane (HFC-245fa) and 1-chloro-3,3,3-trifluoropropene (HFO-1233zd) are compounds that have the potential to be used as zero Ozone Depletion Potential (ODP) and a low Global Warming Potential (GWP) refrigerants, blowing agents, aerosol propellants, insulation agents, solvents, etc., and as starting material for E-1,1,1,3-tetrafluoropropene, HFO-1234ze(E).

U.S. Pat. Nos. 9,045,386 and 5,574,192 describe processes to produce 1-chloro-3,3,3-trifluoropropene (HFO-1233zd) and 1,1,1,3,3-pentafluoropropane (HFC-245fa), respectively, at high purity on a commercial scale. These patents are hereby incorporated herein by reference.

It has been found that certain high boiling residues or by-products are generated in the HFO-1233zd manufacturing process. These by-products are produced in significant volume and uses for same other than disposal are desired.

### SUMMARY

The present disclosure is based on the discovery that that high boiling fluorinated by-products obtained from an HFO-1233zd manufacturing process not only include components that can be used as a starting material or feedstock in the production of HFC-245fa, but also contain impurities that can be detrimental in the HFC-245fa process. The present disclosure is directed toward providing an improved composition from the high boiling by-products obtained from an HFO-1233zd manufacturing process by reducing these impurities. The improved composition can be used as a starting material or feedstock for the production of HFC-245fa and optionally other uses, such as to produce other hydrofluoroolefins.

### BRIEF DESCRIPTION OF THE DRAWINGS

The above mentioned and other features of the disclosure, and the manner of attaining them, will become more apparent and the disclosure itself will be better understood by reference to the following description of embodiments of the disclosure taken in conjunction with the accompanying drawings.

FIG. 1 shows a schematic of a distillation apparatus containing a vaporizer and partial condenser useful for the purification of HFO-1233zd high boiler by-products in order to produce the improved composition.

FIG. 2 shows a schematic of a distillation apparatus containing a vaporizer, distillation tower, and a full condenser useful for the purification of HFO-1233zd high boiler by-products in order to produce the improved composition.

The exemplification set out herein illustrates an embodiment of the disclosure, and such exemplification is not to be construed as limiting the scope of the disclosure in any manner.

### DETAILED DESCRIPTION

The present disclosure is based on the discovery that high boiling fluorinated by-products obtained from an HFO-1233zd manufacturing process not only include components that can be used as a starting material or feedstock in the production of HFC-245fa, but also contain impurities that can be detrimental in the HFC-245fa process. The present disclosure is directed toward providing an improved composition from the high boiling by-products obtained from an HFO-1233zd manufacturing process by reducing these impurities. The improved composition can be used as a starting material or feedstock for the production of HFC-245fa.

HFO-1233zd may be produced industrially by the reaction shown below in Equation 1.

HCC-240fa+3HF→HFO-1233zd+4HCl Equation 1:

In this process, 1-chloro-3,3,3-trifluoropropene (HFO-1233zd) may be produced in a liquid phase reaction without the need for a catalyst. Briefly, anhydrous hydrogen fluoride (HF) may be mixed with 1,1,1,3,3-pentachloropropene (HCC-240fa) at elevated temperature and pressure to produce the desired 1-chloro-3,3,3-trifluoropropene (HFO-1233zd) in a mixture of E- and Z-isomers.

In the course of the reaction, partially fluorinated by-products such as 1,1,3,3-tetrachloro-1-fluoropropane (HCFC-241fa), 1,3,3-trichloro-1,1-difluoropropane (HCFC-242fa), 3,3-dichloro-1,1,1-trifluoropropane (HCFC-243fa), and 3-chloro-1,1,1,3-tetrafluoropropane (HCFC-244fa), isomers of HCFC-241fa, 242fa, 243fa, 244fa, and other minor constituents (such as 1,1,3-tricholoro-1-fluoropropene and 1,3-dichloro-1,1-difluoropropene), may be produced according to Equations 2-5, shown below.

HCC-240fa+HF→HCFC-241fa+HCl Equation 2:

HCC-240fa+2HF→HCFC-242 isomers+2HCl Equation 3:

HCC-240fa+3HF→HCFC-243 isomers+3HCl Equation 4:

HCC-240fa+4HF→HCFC-244 isomers+4HCl Equation 5:

The mixture of high-boiling fluorinated components may be defined as a mixture of high boiling by-products, meaning fluorinated components with boiling points higher than 39° C.; in other words, boiling points higher than that of Z-1-chloro-3,3,3-trifluoropropene (HFO-1233zd(Z)).

Other by-products that may be present in the feed stream or reactor stream include oligomers. Oligomers, as used herein, may be defined as dimers, trimers, tetramers, and other groups of repeated and/or mixed monomeric units that may not be easily converted to 1-chloro-3,3,3-trifluoropropene (HFO-1233zd) using the methods described herein.

Other impurities that may also be present in the reactor as by-products include very high boiling components which, as used herein, mean impurities with boiling points of greater than about 140.2° C., the normal boiling point of HCFC-241fa. These very high boiling components may include CC-110 (hexachloroethane), halogenated hydrocarbons having 5 or more carbon atoms, and some oligomers. For example, possible very high boiling oligomers may be dimers of 1,3,3-trichloro-3-fluoropropene (HCFO-1231zd) and 1,3-dichloro-3,3-difluoropropene (HCFO-1232zd), with the chemical formula C₆H₃F₃Cl₄. The very high boiling components may also include minor olefinic components such as 1,1,3-trichloro-1-fluoropropene and 1,3-dichloro-1,1-difluoropropene. For the purpose of this invention, these very high boiling components, including CC-110, halogenated hydrocarbons having 5 or more carbon atoms, oligomers, and minor olefinic components, may also be referred to as heavy impurities.

Because the process for producing HFO-1233zd and the process for producing HFC-245fa both utilize similar raw materials, the HFO-1233zd process by-products produced by Equations 2-5 are useful as precursors in the production of HFC-245fa. These HFO-1233zd high boiler by-products can be fed into the HFC-245fa reactor together with HF and optionally 1,1,1,3,3-pentachloropropane (HCC-240fa). The ability to use the by-products of the HFO-1233zd process to produce HFC-245fa greatly improves the efficiency of the process.

It has been found that when the high boiling fluorinated by-products are fed into the HFC-245fa reactor, the catalyst activity deteriorates faster than when only HCC-240fa is used. It is believed that the loss of capacity may be caused by impurities including very high boiling fluorinated components and heavy impurities in the HFO-1233zd by-products. In addition, hexachloroethane (CC-110) in particular has been found to cause the formation of solids in the HFC-245fa reactor system that result in a need to shut-down the system periodically to clean it out, with the resultant production losses.

U.S. Pat. No. 5,574,192 discloses the use of the constituents that are present in the HFO-1233zd high boiling by-products in the HFC-245fa manufacturing process to produce HFC-245fa. However, this patent teaches using the components that are present in the HFO-1233zd high boiling by-products in the HFC-245fa manufacturing process without any pre-treatment or purification, such as mitigating very high boiling by-products according to the present teachings. There is no suggestion of the disadvantages, such as loss of catalyst activity, that result from using the HFO-1233zd high boiling by-products in this manner. This reduction in catalyst activity and formation of solids in the liquid phase HFC-245fa process results in loss of production capacity and higher catalyst replacement cost.

The present disclosure mitigates these disadvantages by treating the HFO-1233zd high boiler by-products prior to use in the HFC-245fa reactor to remove or minimize the very high boiling by-products. The treatment transforms the by-product composition into an improved feedstock for HFC-245fa by removing very high-boiling fluorinated components and heavy impurities. The separation of these very high boiling impurities from useful under-fluorinated by-products including 1,3,3-tetrachloro fluoropropane (HCFC-241fa), 1,3,3-trichloro-1,1-difluoropropane (HCFC-242fa), 3,3-dichloro-1,1,1-trifluoropropane (HCFC-243fa), and 3-chloro-1,1,1,3-tetrafluoropropane (HCFC-244fa), along with their isomers. For example, one 242fa isomer is 1,1,3-trichloro-1,3-difluoropropane (HCFC-242fb), and one 243fa isomer is 1,3-dichloro-1,1,3-trifluoropropane (HCFC-243fb). The recycling of these useful by-products allows for more efficient HFO-245fa production without decreasing the catalyst activity or plant capacity for the HFO-245fa reactor.

The present disclosure may be practiced by subjecting the high boiling fluorinated by-products from the production of 1-chloro-3,3,3-trifluoropropene (HFO-1233zd) to a distillation that produces an improved composition comprising isomers of 1,1,3,3-tetrachloro-1-fluoropropane (HCFC-241fa), isomers of 1,3,3-trichloro-1,1-difluoropropane (HCFC-242), isomers of 3,3-dichloro-1,1,1-trifluoropropane (HCFC-243), and isomers of 3-chloro-1,1,1,3-tetrafluoropropane (HCFC-244) with a very low concentration of hexachloroethane and other very high boiling point components having boiling points greater than the boiling point of HCFC-241fa.

The resulting composition, which may be used as a feedstock for production of HCFC-245fa, may comprise at least on component selected from the group consisting of isomers of 1,1,3,3-tetrachloro-1-fluoropropane (HCFC-241fa), isomers of 1,3,3-trichloro-1,1-difluoropropane (HCFC-242), isomers of 3,3-dichloro-1,1,1-trifluoropropane (HCFC-243), and isomers of 3-chloro-1,1,1,3-tetrafluoropropane (HCFC-244) together in an aggregate amount of 50 wt. % or greater, 75 wt. % or greater, or 90 wt. % or greater, based on the total weight of the composition.

The resulting composition, which may be used as a feedstock for production of HCFC-245fa, may also comprise very high-boiling fluorinated components and/or heavy impurities together in an aggregate amount of 5 wt. % or less, 1 wt. % or less, 0.5 wt. % or less, or 0.1 wt. % or less, based on a total weight of the composition.

The resulting composition, which may be used as a feedstock for production of HCFC-245fa, in particular may contain less than 1.0 wt. %, less than 0.5 wt. %, less than 0.3 wt. %, less than 0.1 wt. %, or less than 0.01 wt. % hexachloroethane (CC-110).

In one option, the distillation apparatus may be a two-stage distillation. The apparatus consists of a vaporizer and a partial condenser which separates the HFO-1233zd high boiling components and heavy impurities from the improved composition which is fed into the HFC-245fa reactor.

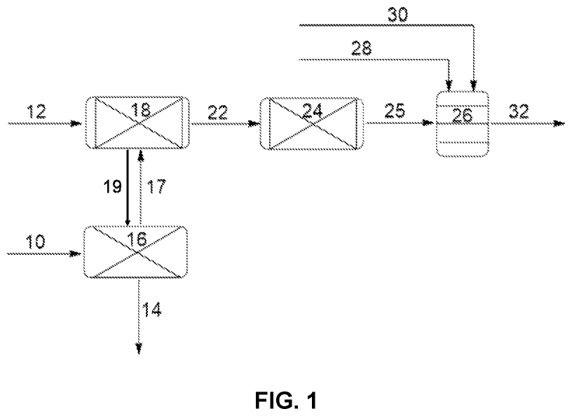

An example of this process is shown in FIG. 1 and summarized below. As shown therein, a stream of the HFO-1233zd high boiling by-products may enter the apparatus as a liquid 10 or, alternatively, as a vapor 12. Feed stream 10 feeds into the partial vaporizer 16 and feed stream 12 feeds into the partial condenser 18.

From vaporizer 16, an effective amount of heat energy is added to the liquid HFO-1233zd high boiling by-products to form vapor stream 17 which is conveyed to the partial condenser 18. Partial condenser 18 is at an effective temperature to partially condense the vapors that enter at the desired pressure to produce a liquid 19 that is returned as reflux to vaporizer 16.

From partial condenser 18, a vapor distillate stream 22 with reduced levels of very high boiling components flows into total condenser 24 and is pumped to provide an improved composition or liquid feedstock stream 25 which, as described above, comprises isomers of 1,1,3,3-tetrachloro-1-fluoropropane (HCFC-241fa), isomers of 1,3,3-trichloro-1,1-difluoropropane (HCFC-242), isomers of 3,3-dichloro-1,1,1-trifluoropropane (HCFC-243), and isomers of 3-chloro-1,1,1,3-tetrafluoropropane (HCFC-244) together with a low level of very high-boiling fluorinated components, heavy impurities, or any other impurities with higher boiling points than HCFC-241fa.

Feedstock stream 25 then enters HFC-245fa reactor 26. HFC-245fa reactor 26 is fed with HCC-240fa from stream 28 and HF from stream 30. The HFC-245fa reactor product 32 may then be removed.

The liquid waste stream 14 comprising very high-boiling fluorinated components, heavy impurities, or any other impurities with higher boiling points than HCFC-241fa may then be removed for disposal or further recovery of useful components.

The vaporizer 16 may be operated at a temperature of 90° C. or greater, 95° C. or greater, 100° C. or greater, 105° C. or greater, 115° C. or greater, 125° C. or greater, 135° C. or greater, 185° C. or lower 175° C. or lower, 165° C. or lower, 155° C. or lower, or within any range encompassing these endpoints.

The partial condenser 18 may be operated at a temperature of 80° C. or greater, 105° C. or greater, 150° C. or greater, 160° C. or greater, 170° C. or lower, 150° C. or lower, 125° C. or lower, 100° C. or lower, or within any range encompassing these endpoints.

The composition in feed stream 10 or 12 may comprise from about 1 wt. % to about 40 wt. % HCFC-241fa isomers, from about 10 wt. % to about 35 wt. % HCFC-241fa isomers, from about 20 wt. % to about 30 wt. % HCFC-241fa isomers, or about 25 wt. % HCFC-241fa isomers. Feed stream 10 or 12 may also comprise from about 40 wt. % to about 80 wt. % of HCFC-242 isomers, from about 54 wt. % to about 74 wt. % of HCFC-242 isomers, from about 59 wt. % to about 69 wt. % HCFC-242 isomers, or about 64 wt. % HCFC-242 isomers. Feed stream 10 or 12 may also comprise from about 21 wt. % or lower HCFC-243 isomers, from about 16 wt. % or lower HCFC-243 isomers, from about 11 wt. % or lower HCFC-243 isomers, or about 6% HCFC-243 isomers. Feed stream 10 or 12 may also comprise from about 15 wt. % or lower HCFC-244 isomers, from about 10 wt. % or lower HCFC-244 isomers, from about 5 wt. % or lower HCFC-244 isomers, or about 0.26 wt. % HCFC-244 isomers. Feed stream 10 or 12 may also comprise from about 15 wt. % or lower hexachloroethane, from about 10 wt. % or lower hexachloroethane, from about 5 wt. % or lower hexachloroethane, or about 0.44 wt. % hexachloroethane.

The improved composition of feed stream 22 may comprise from about 1 wt. % to about 40 wt. % HCFC-241fa isomers, from about 10 wt. % to about 35 wt. % HCFC-241fa isomers, or from about 20 wt. % to about 30 wt. % HCFC-241fa isomers. Feed stream 22 may also comprise from about 40 wt. % to about 80 wt. % of HCFC-242 isomers, from about 54 wt. % to about 74 wt. % of HCFC-242 isomers, or from about 59 wt. % to about 69 wt. % HCFC-242 isomers. Feed stream 22 may also comprise from about 21 wt. % or lower HCFC-243 isomers, from about 16 wt. % or lower HCFC-243 isomers, or from about 11 wt. % or lower HCFC-243 isomers. Feed stream 22 may also comprise from about 15 wt. % or lower HCFC-244 isomers, from about 10 wt. % or lower HCFC-244 isomers, or from about 5 wt. % or lower HCFC-244 isomers.

The improved composition of stream 22 may also comprise very high-boiling fluorinated components and/or heavy impurities together in an amount of 5 wt. % or less, 1 wt. % or less, 0.5 wt. % or less, or 0.1 wt. % or less, based on a total weight of the composition.

The improved composition of stream 22 may also comprise, in particular may contain less than 1.0 wt. %, less than 0.5 wt. %, less than 0.3 wt. %, less than 0.1 wt. %, or less than 0.01 wt. % hexachloroethane (CC-110).

In another option, the distillation apparatus may be a multi-stage distillation. The apparatus includes a tower between the vaporizer and condenser to increase the number of stages of distillation allowing for the desired result to be obtained with less heat energy input to the vaporizer. The tower is equipped with packing or trays that are known and readily available in the industry for carrying out distillations

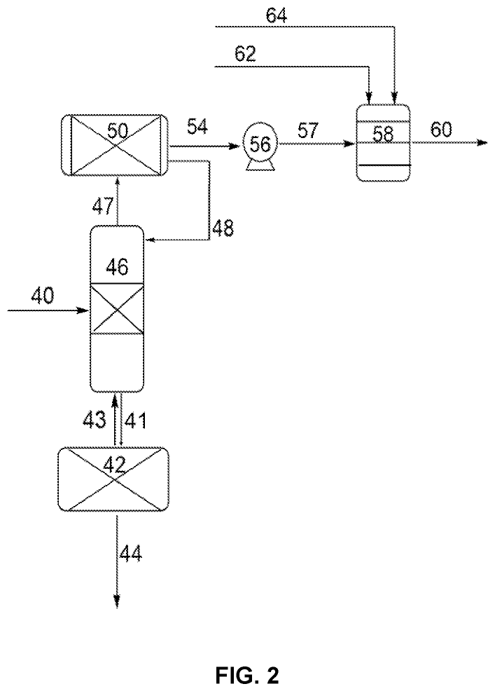

An example of this process is shown in FIG. 2 and summarized below. As shown therein, the HFO-1233zd high boiling by-product feed stream 40 enters the multi-stage distillation process. The addition of distillation tower 46, between the reboiler 42 and condenser 50, allows the condenser 50 to act as a full condenser instead of a partial condenser. From the reboiler, vapor stream 43 enters the distillation tower 46. Reflux from the distillation tower is returned to the reboiler via stream 41. The overhead vapor stream 47 enters the total condenser 50 which operates at an effective temperature to condense the distillate at the desired pressure.

Total condenser 50 provides stream 54. From the total condenser, stream 54 flows into pump 56. The rest of the liquid is returned as reflux to the tower through stream 48.

Pump 56 sends liquid the improved composition or feedstock stream 57 comprising isomers of 1,1,3,3-tetrachloro-1-fluoropropane (HCFC-241fa), isomers of 1,3,3-trichloro-1,1-difluoropropane (HCFC-242), isomers of 3,3-dichloro-1,1,1-trifluoropropane (HCFC-243), and isomers of 3-chloro-1,1,1,3-tetrafluoropropane (HCFC-244), together with a low level of very high-boiling fluorinated components, heavy impurities, or any other impurities with higher boiling points than HCFC-241fa into the HFC-245fa reactor 58 which is fed by feed streams 62 for HF and 64 for HCC-240fa. The HFC-245fa reactor product 60 may then be removed.

The liquid waste stream 44 comprising very high-boiling fluorinated components, heavy impurities, or any other impurities with higher boiling points than HCFC-241fa may then be removed for disposal or further recovery of useful components.

The reboiler 42 may be operated at a temperature of 130° C. or greater, 140° C. or greater, 150° C. or greater, 160° C. or greater, 210° C. or lower 200° C. or lower, 190° C. or lower, 180° C. or lower, or within any range encompassing these endpoints.

The distillation column or tower 46 may be operated at a temperature of 0° C. or greater, 10° C. or greater, 20° C. or greater, 30° C. or greater, 50° C. or lower, 60° C. or lower, 70° C. or lower, 80° C. or lower, or within any range encompassing these endpoints.

The total condenser 50 may be operated at a temperature of 80° C. or greater, 90° C. or greater, 100° C. or greater, 110° C. or greater, 130° C. or lower, 140° C. or lower, 150° C. or lower, 160° C. or lower, or within any range encompassing these endpoints.

The composition in feed stream 40 may comprise from about 10 wt. % to about 40 wt. % HCFC-241 isomers, from about 15 wt. % to about 35 wt. % HCFC-241 isomers, from about 20 wt. % to about 30 wt. % HCFC-241 isomers, or about 25 wt. % HCFC-241 isomers. Feed stream 40 may also comprise from about 49 wt. % to about 79 wt. % of HCFC-242 isomers, from about 54 wt. % to about 74 wt. % of HCFC-242 isomers, from about 59 wt. % to about 69 wt. % HCFC-242 isomers, or about 64 wt. % HCFC-242 isomers. Feed stream 40 may also comprise from about 21 wt. % or lower HCFC-243 isomers, from about 16 wt. % or lower HCFC-243 isomers, from about 11 wt. % or lower HCFC-243 isomers, or about 6% HCFC-243 isomers. Feed stream 40 may also comprise from about 15 wt. % or lower HCFC-244 isomers, from about 10 wt. % or lower HCFC-244 isomers, from about 5 wt. % or lower HCFC-244 isomers, or about 0.26 wt. % HCFC-244 isomers.

Feed stream 40 may also comprise from about 15 wt. % or lower hexachloroethane, from about 10 wt. % or lower hexachloroethane, from about 5 wt. % or lower hexachloroethane, or about 0.44 wt. % hexachloroethane.

Feed stream 40 may also comprise from about 15 wt. % or lower heavy impurities, from about 10 wt. % of lower heavy impurities, from about 5 wt. % or lower heavy impurities, or about 0.24 wt. % heavy impurities.

Feed stream 40 may also comprise from about 20 wt. % or lower of other components, from about 15 wt. % or lower of other components, from about 10 wt. % or lower of other components, or about 4.25 wt. % of other components where these other components may include but are not limited to: 1,1,1,3,3-pentachloropropane (HCC-240fa), isomers of 1,3,3-trichloro-3-fluoro-1-ene (HFO-1231) and 2,3-dichloro-3,3-difluoropropene (HFO-1232), isomers of 1,1,1,3,3,-pentafluoropropane (HFC-245fa), isomers of 1-chloro-3,3,3-trifluoropropene (HFO-1233zd), other impurities including oligomer with boiling points near or lower than 1,1,3,3-tetrachloro-1-fluoropropane (HCFC-241fa).

The improved composition in feed stream 54 may comprise from about 10 wt. % to about 40 wt. % HCFC-241 isomers, from about 15 wt. % to about 35 wt. % HCFC-241 isomers, or from about 20 wt. % to about 30 wt. % HCFC-241 isomers. Feed stream 54 may also comprise from about 49 wt. % to about 79 wt. % of HCFC-242 isomers, from about 54 wt. % to about 74 wt. % of HCFC-242 isomers, or from about 59 wt. % to about 69 wt. % HCFC-242 isomers. Feed stream 54 may also comprise from about 21 wt. % or lower HCFC-243 isomers, from about 16 wt. % or lower HCFC-243 isomers, or from about 11 wt. % or lower HCFC-243 isomers. Feed stream 54 may also comprise from about 15 wt. % or lower HCFC-244 isomers, from about 10 wt. % or lower HCFC-244 isomers, or from about 5 wt. % or lower HCFC-244 isomers.

The improved composition of stream 54 may also comprise very high-boiling fluorinated components and/or heavy impurities together in an aggregate amount of 50 wt. % or less, 25 wt. % or less, 10 wt. % or less, 5 wt. % or less, or 1 wt. % or less, based on a total weight of the composition.

The improved composition of stream 54 may also comprise, in particular may contain less than 1.0 wt. %, less than 0.5 wt. %, less than 0.3 wt. %, less than 0.1 wt. %, or less than 0.01 wt. % hexachloroethane (CC-110).

The distillation in either option can be performed at a pressure of 5 bar or lower, 4 bar or lower, 3 bar or lower, 2 bar or lower, 1 bar or lower, vacuum, or within any range encompassing these endpoints.

The heating system in either option may comprise any suitable heating medium capable of achieving and/or maintaining the temperatures required by the process. Suitable heating media may include molten salt, hot oil, high pressure steam, and electric heaters (resistance or induction), among others, for example.

Fluorination catalysts useful in reactors 26 or 58 include: (I) pentavalent antimony, niobium, arsenic and tantalum halides; (II) pentavalent antimony, niobium, arsenic and tantalum mixed halides; and (III) mixtures of pentavalent antimony, niobium, arsenic and tantalum halide catalysts. Examples of catalysts of group (I) include antimony pentachloride and antimony pentafluoride. Examples of catalysts of group (II) include SbCl₂F₃ and SbBr₂F₃. Examples of catalysts of group (III) include a mixture of antimony pentachloride and antimony pentafluoride.

Other suitable catalysts may include chromium oxides, chromium oxyfluorides, and chromium halides. The chromium oxides may include amorphous chromium oxide (Cr₂O₃), crystalline chromium oxide, and combinations of the foregoing. The chromium oxyfluorides may include fresh amorphous chromium oxide (Cr₂O₃) pretreated with HF, fresh crystalline chromium oxide (Cr₂O₃) pretreated with HF, amorphous chromium oxyfluoride (CrOₓF_{y}, where x may be greater than 0 but less than 1.5, and y may be greater than 0 but less than 3), crystalline chromium oxyfluoride (CrOₓF_{y}, where x may be greater than 0 but less than 1.5, and y may be greater than 0 but less than 3), and combinations of the foregoing. In one embodiment, the catalyst is amorphous chromium oxyfluoride (CrOₓF_{y}, where x may be greater than 0 but less than 1.5, and y may be greater than 0 but less than 3). The chromium halides may include chromium trifluoride (CrF₃), chromium trichloride (CrCl₃), chromium triiodide (CrI₃) and chromium tribromide (CrBr₃), and combinations of the foregoing. In one embodiment, the catalyst is chromium trifluoride (CrF₃).

Other suitable catalysts include promoted chromium-based catalysts, which are based on chromium and include an amount of at least one co-catalyst selected from Ni, Zn, Co, Mn, Mg, or mixtures thereof. The amount of the co-catalyst may be between 0.1 wt. % and 20 wt. % based on the total weight of the catalyst and, more particularly, may be present in an amount as little as 0.1 wt. %, 0.5 wt. %, 1.0 wt. % 1.5 wt. % or as high as 2.0 wt. %, 3.0 wt. %, 4.0 wt. %, 5.0 wt. %, 6.0 wt. %, or within any range using any two of the foregoing values as endpoints, based on to total weight of the catalyst. One suitable promoted chromium catalyst is a zinc/chromia catalyst which is based on chromia and includes an amount of zinc as a cocatalyst, for example, JM 62-3M catalyst available from Johnson Matthey. Prior to use, a fluorination treatment of the catalyst may be conducted using anhydrous HF under conditions effective to convert a portion of metal oxides into corresponding metal fluorides.

The above chromium-based catalysts may also be low chromium (VI) catalysts, having a total content of chromium (VI) oxide in an amount of about 5,000 ppm or less, about 2,000 ppm or less, about 1,000 ppm or less, about 500 ppm or less, about 250 ppm or less, or about 100 ppm or less based on total chromium oxides in the chromium oxide catalyst.

In addition to chromium-based catalysts, other suitable catalysts include alumina, iron oxide, magnesium oxide, zinc oxide, nickel oxide, cobalt oxide, aluminum fluoride or metal fluorides such as iron fluoride, magnesium fluoride, zinc fluoride, nickel fluoride, cobalt fluoride, fluorinated alumina, fluorinated iron oxide, fluorinated magnesium oxide, fluorinated nickel oxide, fluorinated cobalt oxide, titanium fluorides, molybdenum fluorides, aluminum oxyfluorides, and combinations of the foregoing. Prior to use, a fluorination treatment of catalyst containing metal oxide(s) is conducted using anhydrous HF under conditions effective to convert a portion of metal oxide(s) into corresponding metal fluoride(s). Pentavalent antimony, niobium, arsenic and tantalum halides are commercially available, and mixed halides thereof are created in situ upon reaction with HF. Antimony pentachloride is preferred because of its low cost and availability. Pentavalent antimony mixed halides of the formula SbCInF5-n where n is 0 to 5 are more preferred. The fluorination catalysts preferably have a purity of at least about 97%. Although the amount of fluorination catalyst used may vary widely, using from about 5 to about 50%, or preferably from about 10 to about 25% by weight catalyst, relative to the organics is suitable.

### EXAMPLES

### Example 1

### Two-Stage Distillation of High-Boiling Fluorinated HFO-1233zd by-Products

In Example 1, a two-stage distillation was performed using the apparatus outlined in FIG. 1. The distillation experiment was repeated at several pressures and reflux ratios. The results are summarized below in Tables 1-6.

The last entry in each table, “Others”, may include but are not limited to: 1,1,1,3,3-pentachloropropane (HCC-240fa), isomers of 1,3,3-trichloro-3-fluoro-1-ene (HFO-1231) and 2,3-dichloro-3,3-difluoropropene (HFO-1232), isomers of 1,1,1,3,3-pentafluoropropane (HFC-245fa), isomers of 1-chloro-3,3,3-trifluoropropene (HFO-1233zd), other impurities including oligomer with boiling points near or lower than 1,1,3,3-tetrachloro-1-fluoropropane (HCFC-241fa).

TABLE 1
Two-stage distillation at 1 bar and 10:1 reflux ratio.

| | | Improved | High Boilers, |
|---|---|---|---|
| | HFO-1233zd | Feedstock to | Very High |
| | Process High | HFC-245fa | Boilers, and |
| Stream Description/ | Boilers | Reactor | Heavy impurities |
| Components | (Stream 10) | (Stream 22) | (Stream 14) |
| Reflux ratio | | 10 | 10 |
| Fraction of feed | 100% | 97.4% | 2.6% |
| Temperature (° C.) | | 116 | 129 |
| Pressure (bar abs.) | | 1.0 | 1.00 |
| HCFC-241fa isomers | 25.03% | 24.11% | 59.48% |
| HCFC-242 isomers | 63.87% | 64.94% | 23.95% |
| HCFC-243 isomers | 5.90% | 6.06% | 0.23% |
| HCFC-244 isomers | 0.26% | 0.27% | 0.00% |
| Hexachloroethane (110) | 0.44% | 0.26% | 7.10% |
| Oligomer (high BP) | 0.24% | 0.00% | 9.22% |
| Others | 4.25% | 4.37% | 0.00% |

Table 1 summarizes data from a two-stage distillation at 1 bar and 10:1 reflux ratio, with about 2.6% of the feed taken in the bottom stream 14. The temperature of the vaporizer is set to 129° C. At this temperature, most of the high boiling oligomer is in the bottom but only about 43% of the hexachloroethane is in the bottom. Increasing the reflux ratio did not significantly improve the result.

TABLE 2
Two-stage distillation at 1 bar and 10:1 reflux ratio.

| | | | High Boilers, |
|---|---|---|---|
| | | Improved | Very High |
| | HFO-1233zd | Feedstock to | Boilers, and |
| | Process High | HFC-245fa | Heavy impurities |
| Stream Description/ | Boilers | Reactor | to Disposal |
| Components | (Stream 10) | (Stream 22) | (Stream 14) |
| Reflux ratio | | 10 | 10 |
| Fraction of feed | 100% | 79.4% | 20.6% |
| Temperature (° C.) | | 111 | 121 |
| Pressure (bar abs.) | | 1.00 | 1.00 |
| HCFC-241fa isomers | 25.03% | 16.12% | 59.33% |
| HCFC-242 isomers | 63.87% | 70.81% | 37.17% |
| HCFC-243 isomers | 5.90% | 7.34% | 0.38% |
| HCFC-244 isomers | 0.26% | 0.33% | 0.01% |
| Hexachloroethane (110) | 0.44% | 0.05% | 1.95% |
| Oligomer (high BP) | 0.24% | Trace | 1.17% |
| Others | 4.25% | 5.36% | Trace |

Table 2 summarizes data from a two-stage distillation at 1 bar and 10:1 reflux ratio, with about 20% of the feed taken in the bottom stream 14. The temperature of the vaporizer is set to 121° C. At this temperature, most of the high boiling oligomer is in the bottom. Increasing the reflux ratio did not significantly improve the result but increasing the bottom stream to about 20% of the feed resulted in over 90% of the hexachloroethane going to the bottom. In this case, additional recovery of useful materials such as HCFC-241fa isomers and HCFC-242 isomers may be affected by further distilling stream 14, possibly under vacuum to allow for a reduction in temperature.

TABLE 3
Two-stage distillation at 3 bar and 20:1 reflux ratio.

| | | | High Boilers, |
|---|---|---|---|
| | | Improved | Very High |
| | HFO-1233zd | Feedstock to | Boilers, and |
| | Process High | HFC-245fa | Heavy impurities |
| Stream Description/ | Boilers | Reactor | to Disposal |
| Components | (Stream 10) | (Stream 22) | (Stream 14) |
| Reflux ratio | | 20 | 20 |
| Fraction of feed | 100% | 97.4% | 2.6% |
| Temperature (° C.) | | 158 | 172 |
| Pressure (bar abs.) | | 3.00 | 3.00 |
| HCFC-241fa isomers | 25.03% | 24.18% | 56.55% |
| HCFC-242 isomers | 63.87% | 64.84% | 27.90% |
| HCFC-243 isomers | 5.90% | 6.05% | 0.32% |
| HCFC-244 isomers | 0.26% | 0.27% | Trace |
| Hexachloroethane (110) | 0.44% | 0.29% | 6.09% |
| Oligomer (high BP) | 0.24% | Trace | 9.13% |
| Others | 4.25% | 4.37% | Trace |

Table 3 summarizes data from a two-stage distillation at 3 bar and 20:1 reflux ratio, with about 2.6% of the feed removed in the bottom stream 14. The temperature of the vaporizer is set to 172° C. At this temperature, most of the high boiling oligomer is in the bottom but only about 36% of the hexachloroethane is in the bottom. The results suggest that a higher pressure of 3 bar does not favor separation. Compared to the experiments done at 1 bar, the hexachloroethane removal from the bottom was worse, even at a doubled reflux ratio of 20:1.

TABLE 4
Two-stage distillation at 3 bar with 20:1 reflux ratio.

| | | | High Boilers, |
|---|---|---|---|
| | | Improved | Very High |
| | HFO-1233zd | Feedstock to | Boilers, and |
| | Process High | HFC-245fa | Heavy impurities |
| Stream Description/ | Boilers | Reactor | to Disposal |
| Components | (Stream 10) | (Stream 22) | (Stream 14) |
| Reflux ratio | | 20 | 20 |
| Fraction of feed | 100% | 74.9% | 25.1% |
| Temperature (° C.) | | 153 | 163 |
| Pressure (bar abs.) | | 3.00 | 3.00 |
| HCFC-241fa isomers | 25.03% | 15.44% | 53.68% |
| HCFC-242 isomers | 63.87% | 70.79% | 43.21% |
| HCFC-243 isomers | 5.90% | 7.70% | 0.54% |
| HCFC-244 isomers | 0.26% | 0.35% | 0.01% |
| Hexachloroethane (110) | 0.44% | 0.05% | 1.60% |
| Oligomer (high BP) | 0.24% | Trace | 0.97% |
| Others | 4.25% | 5.68% | Trace |

Table 4 summarizes data from a two-stage distillation with at 3 bar and 20:1 reflux ratio, with about 25% of the feed taken in the bottom stream 3. The temperature of the vaporizer is set to 163° C. which is lower than the temperature in Table 3 due to the higher fraction of lower boiling components such as HCFC-242 isomers. In this case, most of the high boiling oligomer is in the bottom. Increasing the reflux ratio did not significantly improve the result but increasing the bottom stream to about 25% of the feed resulted in over 90% of the hexachloroethane going in the bottom. The additional recovery of useful materials (such as HCFC-241fa isomers and HCFC-242 isomers) may be affected by further distilling steam 3, possibly under vacuum to allow for reduction in temperature.

### Example 2

### Multi-Stage Distillation of High-Boiling Fluorinated HFO-1233zd by-Products

In Example 2, a multi-stage distillation was performed using the apparatus outlined in FIG. 2. The distillation experiment was repeated at several pressures and reflux ratios. The results are summarized below in Tables 5 and 6.

The last entry in each table, “Others”, may include but are not limited to: 1,1,1,3,3-pentachloropropane (HCC-240fa), isomers of 1,3,3-trichloro-3-fluoro-1-ene (HFO-1231) and 2,3-dichloro-3,3-difluoropropene (HFO-1232), isomers of 1,1,1,3,3-pentafluoropropane (HFC-245fa), isomers of 1-chloro-3,3,3-trifluoropropene (HFO-1233zd), other impurities including oligomer with boiling points near or lower than 1,1,3,3-tetrachloro-1-fluoropropane (HCFC-241fa).

TABLE 5
Multi-stage distillation at 1 bar and 0.5:1 reflux ratio.

| | | | High Boilers, |
|---|---|---|---|
| | | Improved | Very High |
| | HFO-1233zd | Feedstock to | Boilers, and |
| | Process High | HFC-245fa | Heavy impurities |
| Stream Description/ | Boilers | Reactor | to Disposal |
| Components | (Stream 40) | (Stream 54) | (Stream 44) |
| Reflux ratio | | 0.5 | 0.5 |
| Fraction of feed | 100% | 97.4% | 2.6% |
| Temperature (° C.) | | 101 | 146 |
| Pressure (bar abs.) | | 1.0 | 1.00 |
| HCFC-241fa isomers | 25.03% | 23.72% | 73.74% |
| HCFC-242 isomers | 63.87% | 65.57% | 0.44% |
| HCFC-243 isomers | 5.90% | 6.06% | Trace |
| HCFC-244 isomers | 0.26% | 0.27% | Trace |
| Hexachloroethane (110) | 0.44% | Trace | 16.56% |
| Oligomer (high BP) | 0.24% | Trace | 9.26% |
| Others | 4.25% | 4.37% | Trace |

Table 5 summarizes data from a multi-stage distillation at 1 bar and 0.5:1 reflux ratio, with about 2.6% of the feed taken in the bottom stream 44. The temperature of the vaporizer is set to 146° C., higher than Table 1 because there is less of the lower boiling HCFC-242 isomers. At this temperature, most of the high boiling oligomer and hexachloroethane are in the bottom. While it may be possible to subject the bottom stream 44 to further distillation to recover more of the useful HCFC-241fa isomers, it should be recognized that at a high enough concentration the hexachloroethane and oligomer may form a solid phase if the stream is cooled.

TABLE 6
Multi-stage distillation at 3 bar and 0.5:1 reflux ratio.

| | | | High Boilers, |
|---|---|---|---|
| | | Improved | Very High |
| | HFO-1233zd | Feedstock to | Boilers, and |
| | Process High | HFC-245fa | Heavy impurities |
| Stream Description/ | Boilers | Reactor | to Disposal |
| Components | (Stream 40) | (Stream 54) | (Stream 44) |
| Reflux ratio | | 0.5 | 0.5 |
| Fraction of feed | 100% | 97.4% | 2.6% |
| Temperature (° C.) | | 144 | 193 |
| Pressure (bar abs.) | | 3.00 | 3.00 |
| HCFC-241fa isomers | 25.03% | 23.73% | 73.32% |
| HCFC-242 isomers | 63.87% | 65.56% | 0.95% |
| HCFC-243 isomers | 5.90% | 6.06% | Trace |
| HCFC-244 isomers | 0.26% | 0.27% | Trace |
| Hexachloroethane (110) | 0.44% | 0.01% | 16.46% |
| Oligomer (high BP) | 0.24% | Trace | 9.26% |
| Others | 4.25% | 4.37% | Trace |

Table 6 summarizes data from a multi-stage distillation at 3 bar and 0.5:1 reflux ratio, with about 2.6% of the feed taken in the bottom stream 44. The temperature of the vaporizer is set to 193° C., higher than Table 3 because there is less of the lower boiling HCFC-242 isomers. At this temperature, most of the high boiling oligomer and hexachloroethane are in the bottom. While it may be possible to subject the bottom stream 44 to further distillation to recover more of the useful HCFC-241fa isomers, it should be recognized that at a high enough concentration the hexachloroethane and oligomer may form a solid phase if the stream is cooled.

### Examples 3-27

### Production of HFC-245fa

Feed streams produced in accordance with Examples 1 and 2 are introduced into an HFC-245fa reactor and catalytically reacted with HF to form HFC-245fa at suitable yields and selectivity. The catalysts used in the reaction are set forth below in Table 7 for Examples 3-27:

TABLE 7
Catalysts used in Examples 3-27

| Example | Catalyst |
|---|---|
| 3 | Chromium trifluoride (CrF3) |
| 4 | Chromium trichloride (CrCl₃) |
| 5 | Chromium triiodide (CrI₃) |
| 6 | Chromium tribromide (CrBr₃) |
| 7 | Chromium/zinc with 2.3 wt. % Zn |
| 8 | Chromium oxide with less than 500 ppm chromium (VI) oxide |
| 9 | Alumina |
| 10 | Iron oxide |
| 11 | Magnesium oxide |
| 12 | Zinc oxide |
| 13 | Nickel oxide |
| 14 | Cobalt oxide |
| 15 | Zinc fluoride |
| 16 | Magnesium fluoride |
| 17 | Nickel fluoride |
| 18 | Titanium fluoride |
| 19 | Molybdenum fluoride |
| 20 | Cobalt fluoride |
| 21 | Aluminum fluoride |
| 22 | Aluminum oxyfluoride |
| 23 | Iron fluoride |
| 24 | Fluorinated magnesium oxide |
| 25 | Fluorinated nickel oxide |
| 26 | Fluorinated cobalt oxide |
| 27 | Fluorinated iron oxide |

### ASPECTS

Aspect 1 is a composition useful as a feedstock for the production of HFC-245fa, comprising: at least one compound selected from the group consisting of isomers of 1,1,3,3-tetrachloro-1-fluoropropane (HCFC-241fa), isomers of 1,3,3-trichloro-1,1-difluoropropane (HCFC-242), isomers of 3,3-dichloro-1,1,1-trifluoropropane (HCFC-243), and isomers of 3-chloro-1,1,1,3-tetrafluoropropane (HCFC-244), wherein the foregoing compounds in the aggregate amount to 50 wt. % or greater, based on a total weight of the composition; and at least one very high boiling component, wherein all very high boiling components are present in an aggregate amount of 1 wt. % or less, based on a total weight of the composition.

Aspect 2 is the composition of Aspect 1, wherein the at least one very high boiling component comprises a mixture of fluorinated components with boiling points higher than about 140.2° C.

Aspect 3 is the composition of Aspect 1 or Aspect 2, wherein the at least one very high boiling component comprise at least one of hexachloroethane and halogenated hydrocarbons having 5 or more carbon atoms.

Aspect 4 the composition of any of Aspects 1-3, wherein the at least one very high boiling component is present in an aggregate amount of 0.5 wt. % or less, based on a total weight of the composition.

Aspect 5 is the composition of any of Aspects 1-4, wherein the at least one very high boiling component comprises hexachloroethane in an amount of less than 0.3 wt. %, based on a total weight of the composition.

Aspect 6 is the composition of any of Aspects 1-5, wherein the at least one very high boiling component comprises hexachloroethane in an amount of less than 0.1 wt. %, based on a total weight of the composition.

Aspect 7 is the composition of any of Aspects 1-6, wherein the at least one compound selected from the group consisting of isomers of 1,1,3,3-tetrachloro-1-fluoropropane (HCFC-241fa), isomers of 1,3,3-trichloro-1,1-difluoropropane (HCFC-242), isomers of 3,3-dichloro-1,1,1-trifluoropropane (HCFC-243), and isomers of 3-chloro-1,1,1,3-tetrafluoropropane (HCFC-244) in the aggregate amount to 85 wt. % or greater, based on a total weight of the composition.

Aspect 8 is the composition of any of Aspects 1-7, wherein the at least one compound selected from the group consisting of isomers of 1,1,3,3-tetrachloro-1-fluoropropane (HCFC-241fa), isomers of 1,3,3-trichloro-1,1-difluoropropane (HCFC-242), isomers of 3,3-dichloro-1,1,1-trifluoropropane (HCFC-243), and isomers of 3-chloro-1,1,1,3-tetrafluoropropane (HCFC-244) in the aggregate amount to 95 wt. % or greater, based on a total weight of the composition.

Aspect 9 is a method for producing a composition useful as a feedstock for the production of HFC-245fa, comprising: at least partially condensing a composition comprising at least one compound selected form the group consisting of isomers of 1,1,3,3-tetrachloro-1-fluoropropane (HCFC-241fa), isomers of 1,3,3-trichloro-1,1-difluoropropane (HCFC-242), isomers of 3,3-dichloro-1,1,1-trifluoropropane (HCFC-243), isomers of 3-chloro-1,1,1,3-tetrafluoropropane (HCFC-244) and at least one very high boiling component to form: a vaporized stream comprising the at least one compound selected from the group consisting of isomers of 1,1,3,3-tetrachloro fluoropropane (HCFC-241fa), isomers of 1,3,3-trichloro-1,1-difluoropropane (HCFC-242), isomers of 3,3-dichloro-1,1,1-trifluoropropane (HCFC-243), isomers of 3-chloro-1,1,1,3-tetrafluoropropane (HCFC-244); and a liquid stream comprising the at least one very high boiling component; separating the vaporized stream from the liquid stream; and optionally feeding the vaporized stream to an HFC-245 reactor.

Aspect 10 is the method of Aspect 9, wherein the vaporized stream comprises: at least one compound selected from the group consisting of isomers of 1,1,3,3-tetrachloro-1-fluoropropane (HCFC-241fa), isomers of 1,3,3-trichloro-1,1-difluoropropane (HCFC-242), isomers of 3,3-dichloro-1,1,1-trifluoropropane (HCFC-243), and isomers of 3-chloro-1,1,1,3-tetrafluoropropane (HCFC-244), wherein the foregoing compounds in the aggregate amount to 50 wt. % or greater, based on a total weight of the composition; and at least one very high boiling component, wherein all very high boiling components are present in an aggregate amount of 1 wt. % or less, based on a total weight of the composition.

Aspect 11 is the method of Aspect 9 or Aspect 10, wherein the at least one very high boiling component is present in an aggregate amount of 0.5 wt. % or less, based on a total weight of the vaporized steam.

Aspect 12 is the method of any of Aspects 9-11, wherein the at least one very high boiling component comprises hexachloroethane in an amount of less than 0.3 wt. %, based on a total weight of the vaporized stream.

Aspect 13 is the method of any of Aspects 9-12, wherein the at least one very high boiling component comprises hexachloroethane in an amount of less than 0.1 wt. %, based on a total weight of the vaporized stream.

Aspect 14 is the method of any of Aspects 9-13, wherein the at least one very high boiling component comprises a mixture of fluorinated components with boiling points higher than about 140.2° C.

Aspect 15 is a method for producing a composition useful as a feedstock for the production of HFC-245fa, comprising: distilling a composition comprising at least one compound selected from the group consisting of isomers of 1,1,3,3-tetrachloro-1-fluoropropane (HCFC-241fa), isomers of 1,3,3-trichloro-1,1-difluoropropane (HCFC-242), isomers of 3,3-dichloro-1,1,1-trifluoropropane (HCFC-243), isomers of 3-chloro-1,1,1,3-tetrafluoropropane (HCFC-244), and very high-boiling components to produce a distillate stream and a very high boiling components stream; and optionally feeding the distillate stream to an HFC-245 reactor.

Aspect 16 is the method of Aspect 15, wherein the distillate stream comprises: at least one compound selected from the group consisting of isomers of 1,1,3,3-tetrachloro-1-fluoropropane (HCFC-241fa), isomers of 1,3,3-trichloro-1,1-difluoropropane (HCFC-242), isomers of 3,3-dichloro-1,1,1-trifluoropropane (HCFC-243), and isomers of 3-chloro-1,1,1,3-tetrafluoropropane (HCFC-244), wherein the foregoing compounds in the aggregate amount to 50 wt. % or greater, based on a total weight of the composition; and at least one very high boiling component, wherein all very high boiling components are present in an aggregate amount of 1 wt. % or less, based on a total weight of the distillate stream.

Aspect 17 is the method of Aspect 15 or Aspect 16, wherein the at least one very high boiling component comprises a mixture of fluorinated components with boiling points higher than about 140.2° C.

Aspect 18 is the method of any of Aspects 15-17, wherein the at least one very high boiling component is present in an aggregate amount of 0.5 wt. % or less, based on a total weight of the distillate stream.

Aspect 19 is the method of any of Aspects 15-18, wherein the at least one very high boiling component comprises hexachloroethane in an amount of less than 0.3 wt. %, based on a total weight of the distillate stream.

Aspect 20 is the method of any of Aspects 15-19, wherein the at least one very high boiling component comprises hexachloroethane in an amount of less than 0.1 wt. %, based on a total weight of the distillate stream.

Aspect 21 is the method of any of Aspects 1-20, further comprising the additional step of catalytically reacting the composition to form HFC-245fa using a catalyst selected from alumina, iron oxide, magnesium oxide, zinc oxide, nickel oxide; cobalt oxide; aluminum fluoride, iron fluoride, magnesium fluoride, zinc fluoride, nickel fluoride, cobalt fluoride, fluorinated alumina, fluorinated iron oxide, fluorinated magnesium oxide, fluorinated nickel oxide, fluorinated cobalt oxide, titanium fluoride, molybdenum fluoride, aluminum oxyfluoride, and combinations of the foregoing.

Aspect 22 is the method of any of Aspects 1-20, further comprising the additional step of catalytically reacting the composition to form HFC-245fa using a zinc/chromium catalyst including 0.5 to 6.0 wt. % Zn, based on the total weight of the catalyst.

Aspect 23 is the method of any of Aspects 1-20, further comprising the additional step of catalytically reacting the composition to form HFC-245fa using a chromium catalyst having chromium (VI) oxide in an amount of less than 1,000 ppm, less than 500 ppm, less than 150 ppm or less than 100 ppm.

While this disclosure has been described as relative to exemplary designs, the present disclosure may be further modified within the spirit and scope of this disclosure. Further, this application is intended to cover such departures from the present disclosure as come within known or customary practice in the art to which this disclosure pertains.

1. A composition useful as a feedstock for the production of HFC-245fa, comprising:
at least one compound selected from the group consisting of isomers of 1,1,3,3-tetrachloro-1-fluoropropane (HCFC-241fa), isomers of 1,3,3-trichloro-1,1-difluoropropane (HCFC-242), isomers of 3,3-dichloro-1,1,1-trifluoropropane (HCFC-243), and isomers of 3-chloro-1,1,1,3-tetrafluoropropane (HCFC-244), wherein the foregoing compounds in the aggregate amount to 50 wt. % or greater, based on a total weight of the composition; and
at least one very high boiling component, wherein all very high boiling components are present in an aggregate amount of 1 wt. % or less, based on a total weight of the composition.

2. The composition of claim 1, wherein the at least one very high boiling component comprises a mixture of fluorinated components with boiling points higher than about 140.2° C.

3. The composition of claim 1, wherein the at least one very high boiling component comprise at least one of hexachloroethane and halogenated hydrocarbons having 5 or more carbon atoms.

4. The composition of claim 1, wherein the at least one very high boiling component is present in an aggregate amount of 0.5 wt. % or less, based on a total weight of the composition.

5. The composition of claim 1, wherein the at least one very high boiling component comprises hexachloroethane in an amount of less than 0.3 wt. %, based on a total weight of the composition.

6. The composition of claim 1, wherein the at least one very high boiling component comprises hexachloroethane in an amount of less than 0.1 wt. %, based on a total weight of the composition.

7. The composition of claim 1, wherein the at least one compound selected from the group consisting of isomers of 1,1,3,3-tetrachloro-1-fluoropropane (HCFC-241fa), isomers of 1,3,3-trichloro-1,1-difluoropropane (HCFC-242), isomers of 3,3-dichloro-1,1,1-trifluoropropane (HCFC-243), and isomers of 3-chloro-1,1,1,3-tetrafluoropropane (HCFC-244) in the aggregate amount to 85 wt. % or greater, based on a total weight of the composition.

8. The composition of claim 1, wherein the at least one compound selected from the group consisting of isomers of 1,1,3,3-tetrachloro-1-fluoropropane (HCFC-241fa), isomers of 1,3,3-trichloro-1,1-difluoropropane (HCFC-242), isomers of 3,3-dichloro-1,1,1-trifluoropropane (HCFC-243), and isomers of 3-chloro-1,1,1,3-tetrafluoropropane (HCFC-244) in the aggregate amount to 95 wt. % or greater, based on a total weight of the composition.

9. A method for producing a composition useful as a feedstock for the production of HFC-245fa, comprising:
at least partially condensing a composition comprising at least one compound selected form the group consisting of isomers of 1,1,3,3-tetrachloro-1-fluoropropane (HCFC-241fa), isomers of 1,3,3-trichloro-1,1-difluoropropane (HCFC-242), isomers of 3,3-dichloro-1,1,1-trifluoropropane (HCFC-243), isomers of 3-chloro-1,1,1,3-tetrafluoropropane (HCFC-244) and at least one very high boiling component to form:
a vaporized stream comprising the at least one compound selected from the group consisting of isomers of 1,1,3,3-tetrachloro-1-fluoropropane (HCFC-241fa), isomers of 1,3,3-trichloro-1,1-difluoropropane (HCFC-242), isomers of 3,3-dichloro-1,1,1-trifluoropropane (HCFC-243), isomers of 3-chloro-1,1,1,3-tetrafluoropropane (HCFC-244); and
a liquid stream comprising the at least one very high boiling component;
separating the vaporized stream from the liquid stream; and
optionally feeding the vaporized stream to an HFC-245 reactor.

10. The method of claim 9, wherein the vaporized stream comprises:
at least one compound selected from the group consisting of isomers of 1,1,3,3-tetrachloro-1-fluoropropane (HCFC-241fa), isomers of 1,3,3-trichloro-1,1-difluoropropane (HCFC-242), isomers of 3,3-dichloro-1,1,1-trifluoropropane (HCFC-243), and isomers of 3-chloro-1,1,1,3-tetrafluoropropane (HCFC-244), wherein the foregoing compounds in the aggregate amount to 50 wt. % or greater, based on a total weight of the composition; and
at least one very high boiling component, wherein all very high boiling components are present in an aggregate amount of 1 wt. % or less, based on a total weight of the composition.

11. The method of claim 9, wherein the at least one very high boiling component is present in an aggregate amount of 0.5 wt. % or less, based on a total weight of the vaporized steam.

12. The method of claim 9, wherein the at least one very high boiling component comprises hexachloroethane in an amount of less than 0.3 wt. %, based on a total weight of the vaporized stream.

13. The method of claim 9, wherein the at least one very high boiling component comprises hexachloroethane in an amount of less than 0.1 wt. %, based on a total weight of the vaporized stream.

14. The method of claim 9, wherein the at least one very high boiling component comprises a mixture of fluorinated components with boiling points higher than about 140.2° C.

15. A method for producing a composition useful as a feedstock for the production of HFC-245fa, comprising:
distilling a composition comprising at least one compound selected from the group consisting of isomers of 1,1,3,3-tetrachloro-1-fluoropropane (HCFC-241fa), isomers of 1,3,3-trichloro-1,1-difluoropropane (HCFC-242), isomers of 3,3-dichloro-1,1,1-trifluoropropane (HCFC-243), isomers of 3-chloro-1,1,1,3-tetrafluoropropane (HCFC-244), and very high-boiling components to produce a distillate stream and a very high boiling components stream; and
optionally feeding the distillate stream to an HFC-245 reactor.

16. The method of claim 15, wherein the distillate stream comprises:
at least one compound selected from the group consisting of isomers of 1,1,3,3-tetrachloro-1-fluoropropane (HCFC-241fa), isomers of 1,3,3-trichloro-1,1-difluoropropane (HCFC-242), isomers of 3,3-dichloro-1,1,1-trifluoropropane (HCFC-243), and isomers of 3-chloro-1,1,1,3-tetrafluoropropane (HCFC-244), wherein the foregoing compounds in the aggregate amount to 50 wt. % or greater, based on a total weight of the composition; and
at least one very high boiling component, wherein all very high boiling components are present in an aggregate amount of 1 wt. % or less, based on a total weight of the distillate stream.

17. The method of claim 15, wherein the at least one very high boiling component comprises a mixture of fluorinated components with boiling points higher than about 140.2° C.

18. The method of claim 15, wherein the at least one very high boiling component is present in an aggregate amount of 0.5 wt. % or less, based on a total weight of the distillate stream.

19. The method of claim 15, wherein the at least one very high boiling component comprises hexachloroethane in an amount of less than 0.3 wt. %, based on a total weight of the distillate stream.

20. The method of claim 15, wherein the at least one very high boiling component comprises hexachloroethane in an amount of less than 0.1 wt. %, based on a total weight of the distillate stream.
